(19) United States
(12) Patent Application Publication
(10) Pub. No.: US 2010/0249901 A1
(43) Pub. Date: Sep. 30, 2010

(54) CYLINDRICAL STENT

(75) Inventor: Sung-Gwon Kang, Yongin-Si (KR)

Correspondence Address: Jae Y. Park, Kile, Goekjian, Reed & McManus, PLLC, 1200 New Hampshire Ave. NW, Suite 570, Washington, DC, 20036, US

(73) Assignee: S & G BIOTECH, INC., Seongnam-Si, Gyeonggi-do (KR)

(21) Appl. No.: 12/744,607

(22) PCT Filed: Feb. 15, 2008
(86) PCT No.: PCT/KR08/00897

§ 371 Date: May 25, 2010

(30) Foreign Application Priority Data
Nov. 27, 2007 (KR) 10-2007-0121183

(51) Int. Cl. A61F 2/82

(52) U.S. Cl. 623/1.15

(57) ABSTRACT

The present invention relates to a cylindrical stent, which is a self-expanding type medical appliance for expanding the lumen of a blood vessel or esophagus that is stenosed, more specifically a cylindrical stent made of metal material forming a plurality of peak portions P and valley portions B and having a lengthwise axis. The cylindrical stent is characterized in that the end portions A of the peak portions forming both of the distal portions of the cylindrical stent are bent in the inward direction of the cylindrical stent. The present invention can effectively prevent the problem that a false blood vessel is formed as the vessel wall is exfoliated, broken or damaged due to contact between the distal portion of the cylindrical stent arid the vessel wall, and has an effect of effectively preventing the problem that inflammation occurs at the contact portion due to contact between the lumen wall of an esophagus, etc. and the distal portion of the cylindrical stent.

### TECHNICAL FIELD

The present invention relates to a medical appliance for 5 expanding lumen, and specifically to a self-expanding type medical appliance (hereinafter to be referred to as a “stent”) that expands the lumen of a blood vessel or esophagus that is stenosed. More specifically, the present invention relates to a stent whereby it can effectively prevent the problem that a false blood vessel is formed because the 10 vessel wall is exfoliated, broken or damaged due to contact between the distal portion of a cylindrical stent and the vessel wall, and the problem that inflammation is formed at the contact portion due to contact between the lumen wall of esophagus, etc. and the distal portion of the cylindrical stent.

In general, lumina in the human body have their function lowered because they are stenosed due to diseases that occur in the human body or they cannot function at all in serious cases.

For example, the cases wherein a lumen may become stenosed include that the esophagus is stenosed due to esophageal cancer, that blood is not 20 circulated smoothly due to arteriosclerosis, or that the track for bile to flow from the liver is stenosed.

Because food, blood or bile cannot flow smoothly in such a condition of stenosed lumen, it should be expanded to maintain the passageway. As a method of maintaining the passageway by expanding the stenosed passageway, a stent, for example, is inserted into the lumen.

### BACKGROUND ART

Such a stent has a cylindrical structure in its overall shape and elastic force in itself, so it is widely used since it is contracted by applying external force and expanded if external force is removed. The above-mentioned self-expanding stent should have many characteristics, such as expandability for expanding the lumen, flexibility for flexibly adapting so as to maintain the proper shape of the lumen especially in the case where the lumen is bent, and contractibility for contracting to a given diameter, so as to properly display the function as a stent.

In the aforementioned characteristics of the stent, if expandability is not sufficient, it is easily moved from the initially inserted location; if flexibility is not sufficient, adaptability decreases when the stent expands in the bent portion of the lumen; and if contractibility is not sufficient, the size of the inserting device for inserting the stent increases.

Accordingly, many stents having the aforementioned functions have been developed, and such stents can be classified largely into spiral stents and zigzag stents.

Of the aforementioned spiral stents, the representative one has a cylindrical shape as a whole since it is made by spirally winding a filament having a unit length over and across a cylindrical wall in a zigzag form to and fro between both ends to make a mesh.

Meanwhile, the Korean Patent Laid-Open No. 1998-67399 discloses a stent, wherein the filament makes a plurality of zigzag turns so that the filament makes peak portions and valley portions that connect the straight portions by a plurality of straight portions and a plurality of bending points, and the valley portion of any one turn is made by twisting and connecting the peak portions corresponding to the turn adjacent to this one turn.

The conventional stent mentioned above has a serious problem because the peak portions forming the distal portions on both sides of the stent maintain a straight line with the body of the stent as shown in FIG. 4. In other words, because they are not of a shape bent inward of the stent, a false blood vessel is formed in use as the vessel wall is exfoliated, broken or damaged due to contact between the distal portion of the stent and the vessel wall.

FIG. 4 is a perspective view schematically showing a conventional stent.

Exfoliation of the vessel wall is a phenomenon in which of the three layers composing the vessel wall one inner layer is separated from the other two layers, and this causes a problem of generating a false blood vessel.

In addition, the conventional stent has a serious problem that during its use inflammation occurs at. the contact area between the distal portion of the stent and the lumen wall such as in an esophagus due to the contact.

### DISCLOSURE OF THE INVENTION

### Technical Problem

The present invention is to solve the aforementioned problems of the conventional stent with an object to provide a cylindrical stent, whereby it is possible to effectively prevent the phenomenon that the vessel wall is exfoliated, broken and damaged due to contact with the distal portion of the stent, and prevent inflammation from occurring in the wall of the lumen such as in an esophagus due to contact with the distal portion of the stent.

### Technical Means to Solve the Problem

To solve such problems, there is provided a cylindrical stent wherein the end portions A of the peak portions forming both of the distal portions of the cylindrical stent are bent in the inward direction of the cylindrical stent, that is, in the lengthwise direction of the central axis of the cylindrical stent, so that damage to the vessel wall or lumen wall is prevented by minimizing the contact with the vessel wall or lumen wall when the stent is introduced into the human body.

### ADVANTAGEOUS EFFECTS

The present invention can effectively prevent the problem that a false vessel is formed because the vessel wall is exfoliated, broken or damaged due to contact between the distal portion of a cylindrical stent and the vessel wall, and the problem that inflammation occurs at the contact portion due to contact between the wall of the lumen such as in an esophagus and the distal portion of a cylindrical stent.

### BRIEF DESCRIPTION OF THE DRAWINGS

These and other objects, features, aspects, and advantages of preferred embodiments of the present invention will be more fully described in the following detailed description, taken in conjunction with the accompanying drawings. In the drawings:

FIGS. 1 and 2 are photographs perspectively showing a cylindrical stent according to the present invention.

FIG. 3 is an enlarged photograph of the peak portion forming both of the distal portions of the cylindrical stent according to the present invention.

FIG. 4 is a schematic view of a conventional cylindrical stent.

FIG. 5 is a lengthwise sectional view of a stent manufacturing jig used for manufacturing cylindrical stents according to the present invention.

### DESCRIPTION OF SYMBOLS FOR MAJOR PARTS IN DRAWINGS

P: Peak portion of cylindrical stent

B: Valley portion of cylindrical stent

A: End portion of the peak portion forming both of the distal portions of a cylindrical stent

10: Stent, manufacturing jig

11: Pin of stent manufacturing jig

### BEST MODE FOR CARRYING OUT THE INVENTION

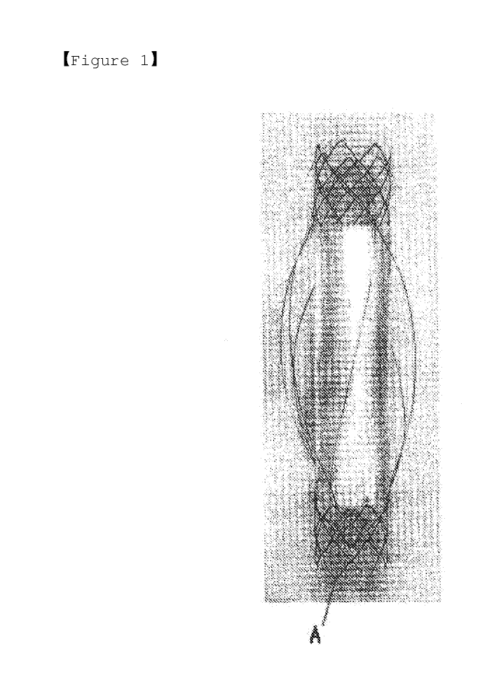

A cylindrical stent according to the present invention is made of metal materials forming a plurality of peak portions P and valley portions B as shown in FIGS. 1 to 3 and has a lengthwise axis. The cylindrical stent is characterized in that the end portions A of the peak portions forming both of the distal portions of the cylindrical stent are bent in the inward direction of the cylindrical stent.

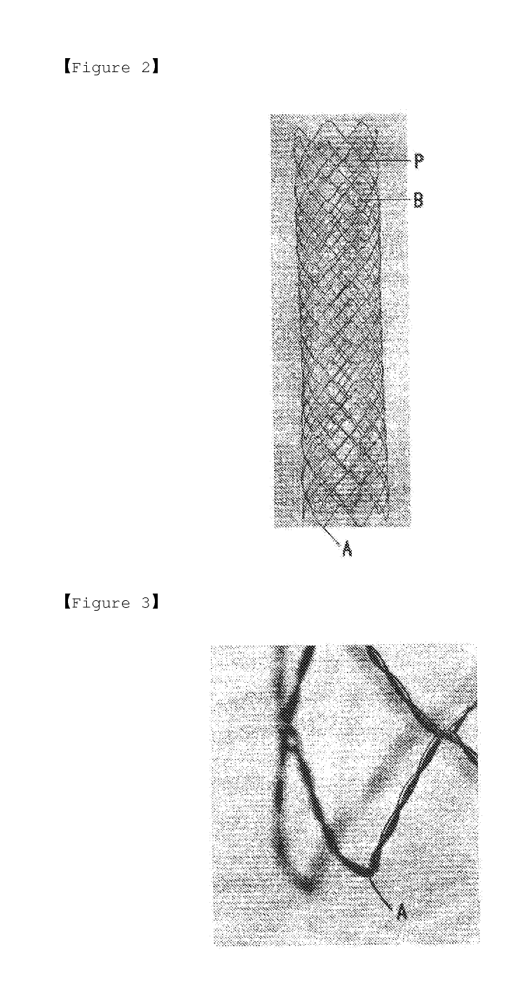

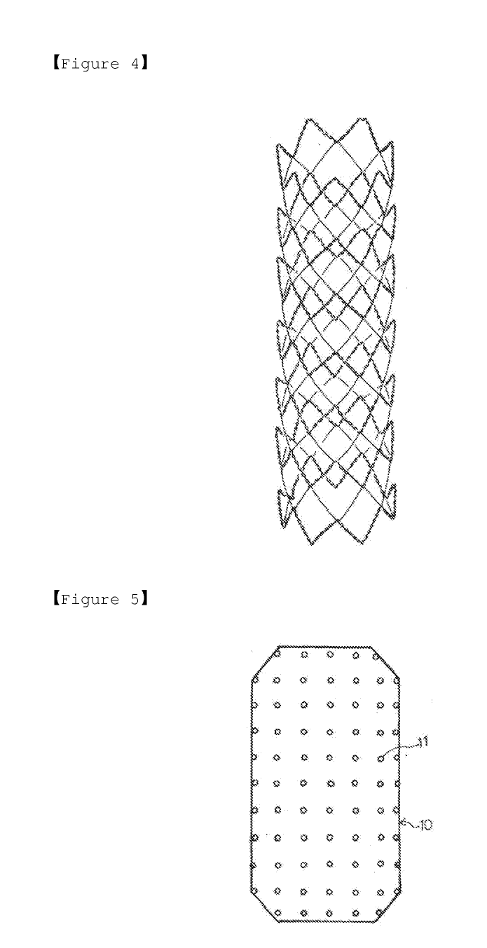

FIG. 1 and FIG. 2 are photographs perspectively showing a cylindrical stent according to the present invention, and FIG. 3 is an enlarged photograph of the peak portions forming both of the distal portions of the cylindrical stent according to the present invention.

In the present invention, that the end portions A of the peak portions forming both of the distal portions of the cylindrical stent are bent in the inward direction of the cylindrical stent means that the end portions A are bent toward the lengthwise central axis of the stent more than the body of the stent.

The cylindrical stent according to the present invention may be made of only the metal material as shown in FIG. 2, and may as well additionally comprise an artificial blood vessel made of fabric as shown in FIG. 1.

Preferably, the metal material is any one selected from a group consisting of nitinol, stainless steel, cobalt chrome, elgiloy, and an alloy thereof, etc.

Preferably, the metal material is any one selected from a group consisting of a wire-cut metal cylinder and a laser-cut metal cylinder.

The cylindrical stent according to the present invention can be manufactured in the usual stent manufacturing process by using a stent manufacturing jig 10 in which the diameter of both distal portions is smaller than the diameter of the central portion as shown in FIG. 5.

FIG. 5 is a lengthwise sectional view of the stent manufacturing jig used for manufacturing the cylindrical stent according to the present invention.

### INDUSTRIAL APPLICABILITY

The present invention is used as a stent for expanding the lumen of a blood vessel or esophagus that is stenosed.

A preferred embodiment of the present invention will now be described in detail with reference to the accompanying drawings. In the following description, well-known functions or constructions are not described in detail since they would obscure the invention in unnecessary detail, and therefore, a detailed description thereof will be omitted.

What is claimed is:

1. A cylindrical stent made of metal material forming a plurality of peak portions P and valley portions B and having a lengthwise axis, wherein the end portions A of the peak portions forming both of the distal portions of said cylindrical stent are characterized by being bent in the inward direction of the cylindrical stent.

2. The cylindrical stent of claim 1, wherein the metal material is any one selected from a group consisting of nitinol, stainless steel, cobalt chrome, elgiloy, and an alloy thereof.

3. The cylindrical stent of claim 1, further comprising an artificial blood vessel made of fabric.

4. The cylindrical stent of claim 1, wherein the metal material is any one selected from a group consisting of a wire-cut metal cylinder and a laser-cut metal cylinder.
